(19) United States
(12) Patent Application Publication
(10) Pub. No.: US 2010/0116271 A1
(43) Pub. Date: May 13, 2010

(54) Apparatus and methods for simultaneous delivery of oxygen and inhaled medication

(76) Inventor: Jonathan Horsford, Atlanta, GA (US)

Correspondence Address: Jonathan Horsford, 5310 Sloan Square NE, Atlanta, GA, 30329, US

(21) Appl. No.: 12/587,435

(22) Filed: Oct. 8, 2009

Related U.S. Application Data
(60) Provisional application No. 61/113,789, filed on Nov. 12, 2008.

(51) Int. Cl. A61M 16/08

(52) U.S. Cl. 128/202.27

(57) ABSTRACT

An adapter for quickly connecting a secondary delivery device to a primary delivery mask is provided. In various embodiments, the adapter includes a hollow body and at least one connection pin. The hollow body includes an open distal end, an open proximal end sized to frictionally mate with an outlet of the secondary delivery device, and an interior chamber extending a length of the body. The at least one connection pin extends from the distal end of the body, substantially parallel with a longitudinal axis of the body. Additionally, the at least one connection pin is structured and sized to frictionally fit within a respective one of a plurality of mask ports formed in the primary delivery mask such that the distal end of the body covers the remaining mask ports.

### FIELD

This application claims priority to Provisional application #61/113,789 filed on Nov. 12, 2008. The present teachings relate to inhaled gas and/or medication delivery devices, and more particularly to an inhaled gas and/or medication delivery device structured and operable for use in combination with an oxygen mask.

### BACKGROUND

The statements in this section merely provide background information related to the present disclosure and may not constitute prior art.

Patients suffering from severe respiratory impairment symptoms or ailments, such as acute severe hypoxemia or acute refractory hypoxemia, often require a high concentration of supplemental oxygen to maintain a life sustaining level of oxygen in the patient's blood. Typically, in order to deliver the appropriate amount of supplemental oxygen to such a patient, an oxygen delivery mask fluidly connected to an oxygen source, is retained over the patient's nose and mouth such that the patient will inhale oxygen flowing from the oxygen source. Exemplary oxygen delivery masks that are commonly used in health care settings, such as hospitals, ambulances, nursing homes, etc., can include what are referred to in the art as “non-rebreather” masks and “venturi/air entrainment” masks.

In some instances, patients may additionally be suffering from a secondary chronic or acute respiratory pathology, such as asthma, pneumonia, or chronic bronchitis, that may be inducing bronchospasm and/or other respiratory distress. Typically, such chronic or acute respiratory pathologies are treated by administering an inhaled medication to relieve the respiratory distress. For example, nebulized or aerosolized liquid medications, i.e., liquid medications mixed with air, can be administered via a hand-held “pipe-shaped” inhaler apparatus or a medication delivery mask similar to an oxygen delivery mask.

However, in the instances where a patient requires administration of a high concentration of oxygen via an oxygen delivery mask, e.g., a non-rebreather mask or a venturi/air entrainment mask, the oxygen delivery mask can not be removed to administer the inhaled medication, lest the patient's oxygen level fall, putting the patient at risk.

### SUMMARY

The present disclosure provides an adapter that is structured and operable to quickly connect a secondary delivery device to a primary delivery mask without substantially interfering with administration of a gas, gas mixture and/or inhaled medication to a patient, via the primary delivery mask. In various embodiments, the adapter includes a hollow body and at least one connection pin. The hollow body includes an open distal end, an open proximal end sized to frictionally mate with an outlet of the secondary delivery device, and an interior chamber extending a length of the body. The at least one connection pin extends from the distal end of the body, substantially parallel with a longitudinal axis of the body. Additionally, the at least one connection pin is structured and sized to frictionally fit within a respective one of a plurality of mask ports formed in the primary delivery mask such that the distal end of the body covers the remaining mask ports.

Further areas of applicability of the present teachings will become apparent from the description provided herein. It should be understood that the description and specific examples are intended for purposes of illustration only and are not intended to limit the scope of the present teachings.

### DRAWINGS

The drawings described herein are for illustration purposes only and are not intended to limit the scope of the present teachings in any way.

FIG. 1 is an isometric view of an oxygen and inhaled medication delivery system including an adaptor structured and operable to quickly couple an inhaled medication delivery device with an oxygen delivery mask for simultaneous delivery and administration of a high concentration of oxygen and an inhaled medication, in accordance with various embodiments of the present disclosure.

FIG. 2 is an exploded isometric view of the system shown in FIG. 1, in accordance with various embodiments of the present disclosure.

FIG. 3 is an angular top view of the adaptor shown in FIG. 1, in accordance with various embodiments of the present disclosure.

FIG. 4 is side view of the adaptor shown in FIG. 1, in accordance with various embodiments of the present disclosure.

Corresponding reference numerals indicate corresponding parts throughout the several views of drawings.

### DETAILED DESCRIPTION

The following description is merely exemplary in nature and is in no way intended to limit the present teachings, application, or uses. Throughout this specification, like reference numerals will be used to refer to like elements.

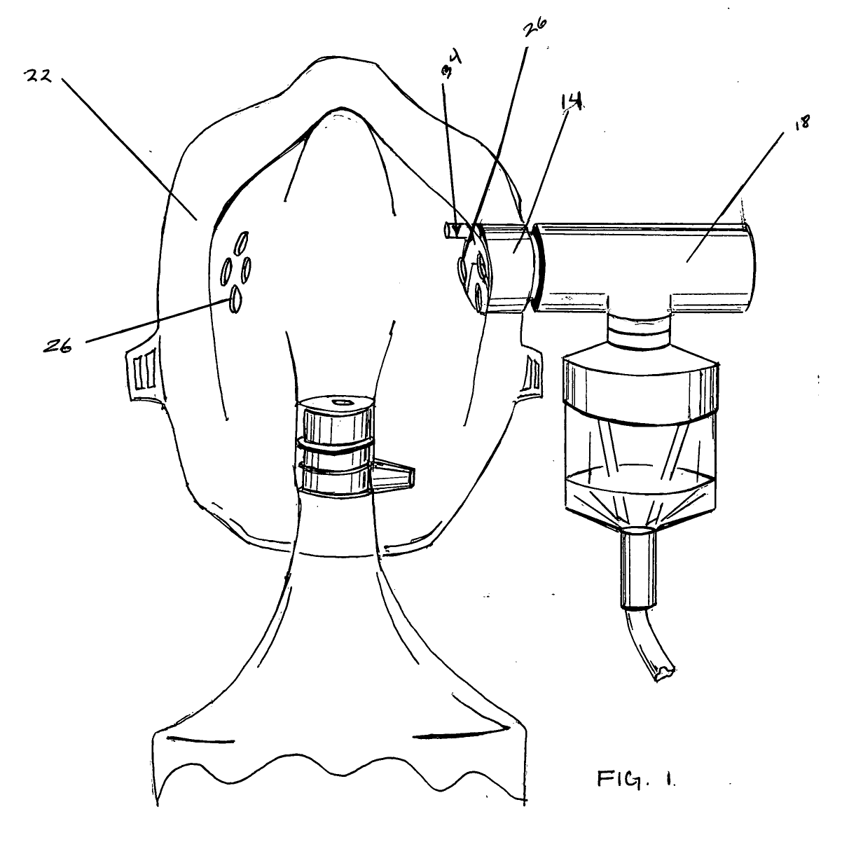

Referring to FIG. 1, the present disclosure provides a breathable gas and/or inhaled medication (BGIM) delivery system 10 structured and operable to simultaneously deliver and administer two or more breathable gases or inhaled medications. For example, in various embodiments, the BGIM delivery system 10 can be utilized to simultaneously deliver and administer a high concentration of oxygen and an inhaled medication.

In various embodiments, the BGIM delivery system 10 includes an adaptor 14 structured and operable to quickly connect a secondary delivery device 18, structured and operable to deliver a secondary breathable gas, gas mixture and/or inhaled medication, with a primary delivery mask 22, or respiratory mask, structured and operable to deliver a controlled concentration of a primary breathable gas or gas mixture. More particularly, the adaptor 14 is structured and operable to quickly connect the secondary delivery device 18 with the primary delivery mask 22 without, or only briefly, removing the primary delivery mask 22 from the face of a respective patient.

The secondary delivery device 18 can be any device, system, apparatus or mechanism structured and operable to deliver to the patient any desired gas, gas mixture and/or inhaled medication, such as oxygen, helium, nitrous oxide, bronchodilators, mixtures thereof, etc. For example, in various embodiments, the secondary delivery device 18 can be an inhaled medication delivery device or system structured and operable to deliver to the patient nebulized or aerosolized liquid medication. More particularly, in various embodiments, the secondary delivery device 18 can comprise a nebulizer device that mixes air with a liquid medication to nebulize the liquid medication. Or, in other embodiments, the secondary delivery device 18 can comprise a metered dose inhaler device that utilizes a compressed propellant to aerosolize a liquid medication.

The primary delivery mask 22 can be any mask that includes a plurality of ports 26 and is structured and operable to deliver to the patient a controlled concentration of a primary breathable gas or gas mixture, such as oxygen, helium, nitrous oxide, mixtures thereof, etc. For example, in various embodiments, the primary delivery mask 22 can be an oxygen delivery mask, such as a rebreather mask or a venturi/air entrainment mask, that includes a plurality of exhalation ports 26 and is structured and operable to fluidly connect to an oxygen source (not shown) for delivering a high concentration of oxygen to the patient. Therefore, in various embodiments, the adaptor 14 can be used to simultaneously deliver and administer to the patient an inhaled medication, e.g., a nebulized or aerosolized medication, via the secondary delivery device 18, and a high concentration of oxygen, via the primary delivery mask 22.

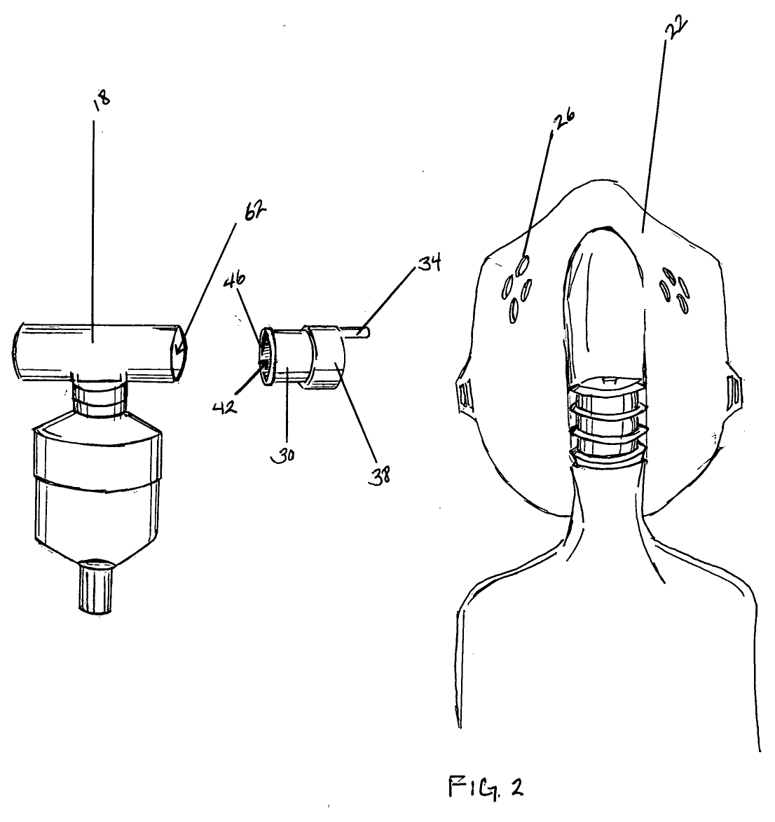

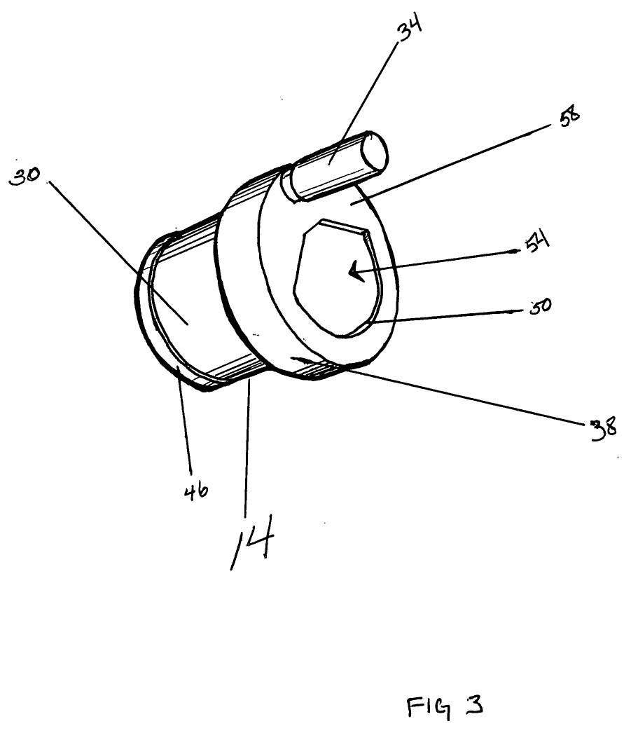

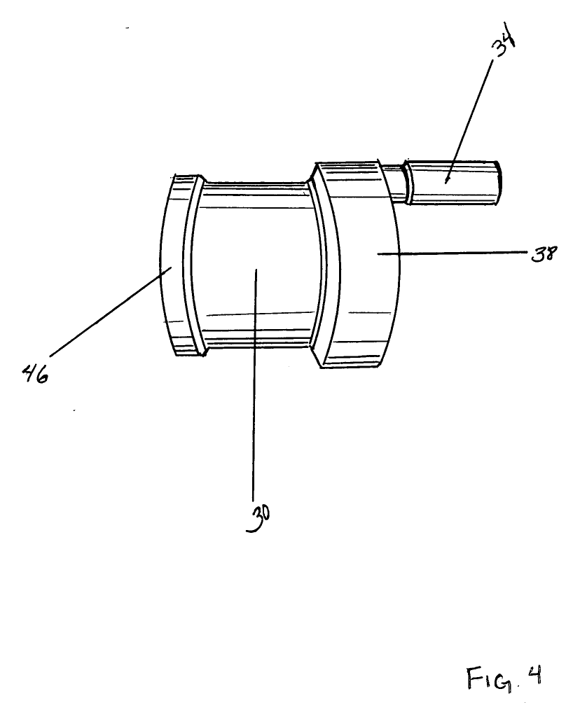

Referring now to FIGS. 2, 3 and 4, in various embodiments, the adaptor 14 includes a hollow, e.g., cylindrical, body 30 having one or more connector pins 34 longitudinally extending from a distal end 38 of the body 30 parallel to an axis X of the body 30. The body 30 includes an open interior chamber 42 extending the length of the body 30 such that gases and/or nebulized/aerosolized medication can flow through the body 30 from a proximal end 46 to the distal end 38.

In various embodiments, the adapter 14 can include a lip 50 formed along at least a portion of the distal end 38 and extending radially inward such that the lip 50 covers a portion of the open distal end of the interior chamber 42 forming a window, or opening, 54 at the distal end 38. In such embodiments, the connector pin(s) 34 can extend orthogonally from the lip 42 parallel to the axis X. Alternatively, in various embodiments, the pin(s) 34 can be integrally formed with a wall 58 of the body 30, along an interior or exterior surface thereof, and extend longitudinally from the body distal end 38 parallel with the axis X, thereby leaving the open distal end of the interior chamber 42 substantially unobstructed.

The proximal end 46 of the body 30 is sized to mate with and frictionally couple with an outlet 62 of the secondary delivery device 18, e.g., an outlet end of T-piece of a small volume nebulizer. Accordingly, a gas, gas mixture and/or inhaled medication delivered by the secondary delivery device 18 will exit the secondary delivery device outlet 62 and enter the interior chamber 42 of the adapter body 30. More particularly, the gas, gas mixture and/or inhaled medication will exit the secondary delivery device outlet 62, enter the interior chamber 42, flow through the interior chamber 42 and exit the interior chamber 42 via the open distal end or opening 54.

The connector pin(s) 34 is/are formed to have an outer diameter D and a length L sized to frictionally couple each pin with a respective one of the primary delivery mask ports 26. That is, the outer diameter D of each connector pin 34 is sized such that each connector pin 34 can be inserted into, i.e., pushed through, a respective primary delivery mask port 26 and be frictionally retained therein. For example, the outer diameter D of each connector pin 34 can be substantially equal to, or slightly larger than an inside diameter of each respective primary delivery mask port 26. Additionally, the length L of each connector pin 34 is sized such that once each connector pin 34 is inserted into, and frictionally coupled with, the respective primary delivery mask port 26, the adaptor 14 will be securely coupled to the primary delivery mask 22 and is resistant to unintentional disconnection due to movement, jarring or shaking of the primary delivery mask 22 and/or the secondary delivery device 18.

Importantly, the distal end 38 of the adaptor body 30 is sized such that when the adaptor 14 is coupled to the primary delivery mask 22, as described above, the open distal end of the interior chamber 42, or the opening 54, covers the remaining mask ports 26. Thus, in instances where a primary gas or gas mixture is being administered to a patient via the primary delivery mask 22, and administration of a secondary gas, gas mixture and/or inhaled medication is desired without, or only briefly, disturbing administration of the primary gas or gas mixture, the adaptor 14 can be utilized to deliver the secondary gas, gas mixture and/or inhaled medication through the mask ports 26.

Particularly, to administer the secondary gas, gas mixture and/or inhaled medication in such instances, the proximal end 46 of adaptor body 30 is frictionally coupled to the outlet 62 of the secondary delivery device 18 and the distal end 38 of the adapter 14 is coupled to the primary delivery mask 22 by inserting each connection pin 34 into a respective one of the mask ports 26 such that the open distal end, or opening 54, of the interior chamber 42 covers the remaining mask ports 26. Thereafter, the secondary gas, gas mixture and/or inhaled medication can be delivered to the proximal end 46 of the adapter body 30, whereby the secondary gas, gas mixture and/or inhaled medication will flow though the adaptor interior chamber 42 and enter the primary delivery mask 22, via the mask ports 26. Accordingly, the secondary gas, gas mixture and/or inhaled medication will mix with the primary gas or gas mixture inside the primary delivery mask 22 and be administered to the patient without, or only briefly, disturbing the administration of the primary gas or gas mixture.

It is envisioned that the connection of the primary delivery mask 22 and the secondary delivery device 18 to the adaptor 14 can be made in any order, i.e., connecting the secondary delivery device 18 to the adapter first and then subsequently connecting the adapter 14 to the primary delivery mask 22, or vice-versa. Additionally, it should be understood that the adapter 14 can be quickly connected to the primary delivery mask 22, as described herein, without, or only briefly, removing the primary delivery mask 22 from the face of a respective patient. Therefore the secondary gas, gas mixture and/or inhaled medication can be administered without, or only briefly, disturbing the administration of the primary gas or gas mixture. For example, the adapter 14, as described herein, can be utilized to administer an inhaled medication to a severely hypoxemic patient receiving a high concentration of oxygen, via a rebreather mask, without interfering with the administration of oxygen.

The description herein is merely exemplary in nature and, thus, variations that do not depart from the gist of that which is described are intended to be within the scope of the teachings. Such variations are not to be regarded as a departure from the spirit and scope of the teachings.

What is claimed is:

1. An adapter for a primary gas or inhaled medication delivery mask, said adapter comprising a hollow body including:
an open distal end;
an open proximal end sized to frictionally mate with an outlet of a secondary gas or inhaled medication delivery device; and
an interior chamber extending a length of the body, wherein the adapter is structured and operable to connect the secondary delivery device to the primary delivery mask without substantially interfering with administration of a gas, gas mixture and/or inhaled medication to a patient, via the primary delivery mask.

2. The adapter of claim 1, wherein the adapter further comprises at least one connection pin extending from the distal end of the body, substantially parallel with a longitudinal axis of the body, the at least one connection pin structured and sized to frictionally fit within a respective one of a plurality of mask ports formed in the primary delivery mask such that the distal end of the body covers the remaining mask ports.

3. The adapter of claim 2, wherein the at least one connection pin is structured to have an outer diameter and a length sized to frictionally couple with the respective one of the plurality of the mask ports.

4. The adapter of claim 2, wherein the adapter further comprises a lip formed along at least a portion of the open distal end and extending radially inward forming an open window at the open distal end, wherein the at least one connector pin extends orthogonally from the lip.

5. The adapter of claim 2, wherein the at least one connector pin is integrally formed with a wall of the body and extends longitudinally from the body distal end.

6. The adapter of claim 5, wherein the at least one connector pin extends along an interior of the body wall.

7. The adapter of claim 5, wherein the at least one connector pin extends along an exterior of the body wall.

8. The adapter of claim 1, wherein the primary deliver mask comprises a non-rebreather mask.

9. The adapter of claim 1, wherein the primary deliver mask comprises a venturi/air entrainment mask.

10. The adapter of claim 1, wherein the secondary delivery device comprises a device structured and operable to deliver a nebulized inhaled medication.

11. The adapter of claim 1, wherein the secondary delivery device comprises a device structured and operable to deliver an aerosolized inhaled medication.

12. The adapter of claim 1, wherein the secondary delivery device comprises a device structured and operable to deliver an inhaled breatheable gas.
